(19) United States
(12) Patent Application Publication
(10) Pub. No.: US 2012/0311728 A1
(43) Pub. Date: Dec. 6, 2012

(54) METHODS AND PHARMACEUTICAL COMPOSITION FOR THE TREATMENT OF ATHEROSCLEROSIS

(76) Inventors: Ziad Mallat, Paris (FR); Gerard Lambeau, Valbonne Sophia Antipolis (FR)

(21) Appl. No.: 13/508,152

(22) PCT Filed: Nov. 5, 2010
(86) PCT No.: PCT/EP2010/066864

§ 371 Date: Aug. 27, 2012

(30) Foreign Application Priority Data
Nov. 6, 2009 (EP) 09306064

(51) Int. Cl. A61K 38/17; C12Q 1/68; A61P 9/10; A01K 67/00; A61K 31/711; A61K 35/00

(52) U.S. Cl. 800/9; 514/1.9; 514/44 R; 424/93.2; 435/6.11

(57) ABSTRACT

The present invention relates to the prevention or treatment of atherosclerosis, in particular to a group X sPLA2 polypeptide for use in the treatment of atherosclerosis.

### FIELD OF THE INVENTION

The present invention relates to the prevention or treatment of atherosclerosis.

### BACKGROUND OF THE INVENTION

Atherosclerosis is the most common cause of death in western societies and is predicted to become the leading cause of cardiovascular disease in the world within two decades. Atherosclerosis contributes to the development of atherosclerotic vascular diseases (AVD) which may affect the coronary arteries (causing ischaemic heart disease), the cerebral circulation (causing cerebrovascular disease), the aorta (producing aneurysms that are prone to thrombosis and rupture) and peripheral blood vessels, typically the legs (causing peripheral vascular disease and intermittent claudication). Ischaemic heart disease (IHD) includes angina (chest pain caused by insufficient blood supply to cardiac muscle) and myocardial infarction (death of cardiac muscle) and cerebrovascular disease includes stroke and transient ischaemic attacks. One in three men and one in four women will die from IHD and the death rate for IHD was 58 per 100,000 in 1990.

So, there is a recognized and permanent need in the art for new reliable methods for treating atherosclerosis.

The different steps of atherogenesis comprise activation and dysfunction of endothelial cells, adhesion, migration and activation of leukocytes in the vascular wall, subendothelial lipoprotein retention and modification into proatherogenic particles, transformation of monocytes into macrophage foam cells and deposition of atheromatous lipids. Following the accumulation of additional inflammatory cell subsets and extracellular lipids, the early atherosclerotic plaques then progress into mature plaques. These latter can become necrotic, fibrous, ultimately resulting in plaque rupture, and leading to arterial occlusion and myocardial infarction or stroke.

Secreted phospholipases A2 (sPLA2s, 14-20 kDa) constitute a relatively novel family of enzymes which exert multiple biological functions, in particular in host immunity, cancer, as well as in several acute and chronic inflammatory diseases. They are distinct in structure from the different intracellular sPLA2s. sPLA2s are expressed and released by numerous inflammatory cells and generate arachidonic acid and lysophospho lipids, thus contributing to the production of numerous bioactive lipid mediators. sPLA2s, notably III, V, X can also efficiently hydrolyze lipoproteins, thereby producing various fatty acids and lysophosphatidylcholine as well as generating a modified LDL particle which is pro-atherogenic {Karabina, 2006}.

There is now accumulating evidence that several sPLA2s, notably IIA, III, V and X play different non redundant roles in one or several steps of atherogenesis {Ghesquiere, 2005; Karabina, 2006; Jonsson-Rylander, 2008; Boyanovsky, 2008; Sato, 2008; Curfs, 2008}. sPLA2s may exert multiple proatherogenic effects in the arterial wall by generating proinflammatory bioactive lipid mediators, by hydrolyzing LDL and generating pro-atherogenic LDL particles, and by activating different inflammatory cells by catalytically-independent mechanisms. There is also a number of epidemiological evidence on the association between increased mass or elevated activity of sPLA2 and risk of cardiovascular disease {Mallat, 2005; Mallat, 2007; Koenig, 2008}. The different studies performed in vitro and in vivo in mouse models of atherosclerosis all converge to the proposal that the sPLA2s IIA, III, V and X may be pro-atherogenic, yet their molecular mechanisms of action is likely to be different {Ivandic, 1999; Tietge, 2005; Webb, 2003; Ghesquiere, 2005; Karabina, 2006; Bostrom, 2007; Curfs, 2008; Fujioka, 2008; Sato, 2008; Curfs, 2008; Boyanovsky, 2009}.

Accordingly, numerous pharmaceutical companies have been interested in these enzymes and have developed high affinity competitive inhibitors against the prototypic group IIA sPLA2 which is over-expressed in numerous human inflammatory diseases {Vadas, 1993; Nevalainen, 2000; Menschikowski, 2006}.

### SUMMARY OF THE INVENTION

The present invention relates to a group X sPLA2 polypeptide for use in the treatment of atherosclerosis.

The present invention also relates to a nucleic acid molecule encoding for a group X sPLA2 polypeptide for use in the treatment of atherosclerosis.

The present invention also relates to a vector comprising a nucleic acid molecule encoding for a group X sPLA2 polypeptide for use the treatment of atherosclerosis.

The present invention also relates to a host cell comprising a nucleic acid molecule encoding for a group X sPLA2 polypeptide or a vector comprising a nucleic acid thereof for use in the treatment of atherosclerosis.

The present invention also relates a non human animal model for atherosclerosis wherein said atherosclerosis is induced by inhibiting the expression or activity of group X sPLA2.

The present invention also relates to a method of testing a subject thought to have or be predisposed to having atherosclerosis, which comprises the step of analyzing a biological sample from said subject for:

(i) detecting the presence of a mutation in the gene encoding for group X sPLA2 and/or its associated promoter, and/or

(ii) analyzing the expression of the gene encoding for group X sPLA2 and/or

(iii) analysing the activity of group X sPLA2.

### DETAILED DESCRIPTION OF THE INVENTION

The inventors surprisingly found that group X sPLA2 is likely to exert multiple roles in atherogenesis which are in fact manifested in vivo in LDL receptor-deficient mice by a protective effect rather than a pro-atheroclerotic one. More particularly, the inventors demonstrate that mouse group X sPLA2 expressed by bone marrow-derived cells reduces mononuclear cell adhesion to atherosclerotic vessels, limits lesional T cell accumulation and their activation towards a pathogenic Thl profile and reduces apoptotic cell accumulation. As a consequence, group X sPLA2 expression limits the development of atherosclerosis and the enlargement of the necrotic lipid core.

Methods of Treatment:

Accordingly a first aspect of the present invention relates to a method for treating atherosclerosis in a patient in need thereof comprising the step of administrating said patient with a group X sPLA2 polypeptide.

The method according to the present invention can be supplied to a patient, which has been diagnosed as presenting one of the following coronary disorders:
asymptomatic coronary artery coronary diseases with silent ischemia or without ischemia;
chronic ischemic disorders without myocardial necrosis, such as stable or effort angina pectoris;
acute ischemic disorders myocardial necrosis, such as unstable angina pectoris;
ischemic disorders with myocardial necrosis, such as ST segment elevation myocardial infarction or non-ST segment elevation myocardial infarction.

In the context of the invention, the term “treating” or “treatment”, as used herein, means reversing, alleviating, inhibiting the progress of, or preventing the disorder or condition to which such term applies, or one or more symptoms of such disorder or condition. A “therapeutically effective amount” is intended for a minimal amount of active agent which is necessary to impart therapeutic benefit to a subject. For example, a “therapeutically effective amount” to a patient is such an amount which induces, ameliorates or otherwise causes an improvement in the pathological symptoms, disease progression or physiological conditions associated with a disorder or resistance to succumbing to a disorder.

The term “patient” refers to any subject (preferably human) afflicted with or susceptible to be afflicted with atherosclerosis.

The term “group X sPLA2 polypeptide” has its general meaning in the art and includes naturally occurring group X secreted phospholipase A2 and conservative function variants and modified forms thereof The group X sPLA2 polypeptide can be from any source, but typically is a mammalian (e.g., human and non-human primate) group X sPLA2 polypeptide, and more particularly a human group X sPLA2 polypeptide.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, most preferably at least 85%, and even more preferably at least 90%, and which has the same or substantially similar properties or functions as the native or parent protein to which it is compared.

In specific embodiments, it is contemplated that group X sPLA2 polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consists of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 45 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology Linking group libraries for use in activated drug delivery are known to those of skill in the art and may be based on enzyme kinetics, prevalence of active enzyme, and cleavage specificity of the selected disease-specific enzymes (see e.g., technologies of established by VectraMed, Plainsboro, N.J.). Such linkers may be used in modifying the group X sPLA2-derived proteins described herein for therapeutic delivery.

According to the invention, group X sPLA2 polypeptides of the invention may be produced by conventional automated peptide synthesis methods or by recombinant expression. General principles for designing and making proteins are well known to those of skill in the art.

Group X sPLA2 polypeptides of the invention may be synthesized in solution or on a solid support in accordance with conventional techniques. Various automatic synthesizers are commercially available and can be used in accordance with known protocols. Group X sPLA2 polypeptides of the invention may also be synthesized by solid-phase technology employing a peptide synthesizer. The purity of any given protein; generated through automated peptide synthesis or through recombinant methods may be determined using reverse phase HPLC analysis. Chemical authenticity of each peptide may be established by any method well known to those of skill in the art.

As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides.

A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Those of skill in the art are aware of various techniques for optimizing mammalian expression of proteins. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and are briefly described herein below. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the recombinant production of the group X sPLA2 polypeptides of the invention, it would be necessary to employ vectors comprising polynucleotide molecules for encoding the group X sPLA2-derived proteins. Methods of preparing such vectors as well as producing host cells transformed with such vectors are well known to those skilled in the art. The polynucleotide molecules used in such an endeavor may be joined to a vector, which generally includes a selectable marker and an origin of replication, for propagation in a host. These elements of the expression constructs are well known to those of skill in the art. Generally, the expression vectors include DNA encoding the given protein being operably linked to suitable transcriptional or translational regulatory sequences, such as those derived from a mammalian, microbial, viral, or insect genes. Examples of regulatory sequences include transcriptional promoters, operators, or enhancers, mRNA ribosomal binding sites, and appropriate sequences which control transcription and translation.

The terms “expression vector,” “expression construct” or “expression cassette” are used interchangeably throughout this specification and are meant to include any type of genetic construct containing a nucleic acid coding for a gene product in which part or all of the nucleic acid encoding sequence is capable of being transcribed.

The choice of a suitable expression vector for expression of the peptides or polypeptides of the invention will of course depend upon the specific host cell to be used, and is within the skill of the ordinary artisan. Methods for the construction of mammalian expression vectors are disclosed, for example, in EP-A-0367566; and WO 91/18982.

Expression requires that appropriate signals be provided in the vectors, such as enhancers/promoters from both viral and mammalian sources that may be used to drive expression of the nucleic acids of interest in host cells. Usually, the nucleic acid being expressed is under transcriptional control of a promoter. A “promoter” refers to a DNA sequence recognized by the synthetic machinery of the cell, or introduced synthetic machinery, required to initiate the specific transcription of a gene. Nucleotide sequences are operably linked when the regulatory sequence functionally relates to the DNA encoding the protein of interest (i.e., group X sPLA2, a variant and the like). Thus, a promoter nucleotide sequence is operably linked to a given DNA sequence if the promoter nucleotide sequence directs the transcription of the sequence.

Similarly, the phrase “under transcriptional control” means that the promoter is in the correct location and orientation in relation to the nucleic acid to control RNA polymerase initiation and expression of the gene. Any promoter that will drive the expression of the nucleic acid may be used. The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerol kinase promoter and glyceraldehyde-3-phosphate dehydrogenase promoter, all of which are promoters well known and readily available to those of skill in the art, can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient to produce a recoverable yield of protein of interest. By employing a promoter with well known properties, the level and pattern of expression of the protein of interest following transfection or transformation can be optimized. Inducible promoters also may be used.

Another regulatory element that is used in protein expression is an enhancer. These are genetic elements that increase transcription from a promoter located at a distant position on the same molecule of DNA. Where an expression construct employs a cDNA insert, one will typically desire to include a polyadenylation signal sequence to effect proper polyadenylation of the gene transcript. Any polyadenylation signal sequence recognized by cells of the selected transgenic animal species is suitable for the practice of the invention, such as human or bovine growth hormone and SV40 polyadenylation signals.

Another aspect of the invention relates to a method for treating atherosclerosis in a patient in need thereof comprising the step of administrating said patient with a nucleic acid molecule encoding for a group X sPLA2 polypeptide.

Typically, said nucleic acid is a DNA or RNA molecule, which may be included in any suitable vector, such as a plasmid, cosmid, episome, artificial chromosome, phage or a viral vector as above described.

So, a further object of the invention relates to a method for treating atherosclerosis in a patient in need thereof comprising the step of administrating said patient with a vector comprising a nucleic acid molecule encoding for a group X sPLA2 polypeptide.

A further object of the invention relates to a method for treating atherosclerosis in a patient in need thereof comprising the step of administrating said patient with a host cell comprising a nucleic acid molecule encoding for a group X sPLA2 polypeptide (or a vector comprising a nucleic acid thereof).

The group X sPLA2 polypeptide of the invention (or nucleic acids, vectors or host cells according to the invention) may be administered in the form of a pharmaceutical composition, as defined below.

Preferably, said group X sPLA2 polypeptide (or nucleic acids, vectors or host cells according to the invention) is administered in a therapeutically effective amount.

By a “therapeutically effective amount” is meant a sufficient amount of the group X sPLA2 polypeptide (or nucleic acids, vectors or host cells according to the invention) to treat or to prevent atherosclerosis at a reasonable benefit/risk ratio applicable to any medical treatment.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved.

A further aspect of the invention relates to a method for preventing a vascular or coronary disorder comprising the step of administrating a patient in need thereof with a group X sPLA2 polypeptide (or a nucleic acid, vector or host cell according to the invention).

In a particular embodiment, said coronary disorder or vascular disorders is selected from the group consisting of atherosclerotic vascular disease, such as aneurysm or stroke, asymptomatic coronary artery diseases, chronic ischemic disorders without myocardial necrosis, such as stable or effort angina pectoris; acute ischemic disorders myocardial necrosis, such as unstable angina pectoris; and ischemic disorders such as myocardial infarction.

Pharmaceutical Compositions:

The group X sPLA2 polypeptide of the invention (or nucleic acids, vectors or host cells according to the invention) may be combined with pharmaceutically acceptable excipients, and optionally sustained-release matrices, such as biodegradable polymers, to form pharmaceutical compositions.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The group X sPLA2 polypeptide of the invention (or nucleic acids, vectors or host cells according to the invention) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

Non Human Animal Model for Atherosclerosis:

A further object of the invention relates a non human animal model for atherosclerosis wherein said atherosclerosis is induced by inhibiting the expression or activity of group X sPLA2.

In a particular embodiment, inhibiting the activity of group X sPLA2 may be performed with an inhibitor which may consist in a small organic molecule, aptamers, or antibodies or either any compound natural or not capable of inhibiting the activity of group X sPLA2.

In a particular embodiment, inhibiting the expression of group X sPLA2 may be performed with an inhibitor of gene expression.

An “inhibitor of gene expression” refers to a natural or synthetic compound that has a biological effect to inhibit or significantly reduce the expression of a gene.

Inhibitors of gene expression for use in the present invention may be also based on anti-sense oligonucleotide constructs. Anti-sense oligonucleotides, including anti-sense RNA molecules and anti-sense DNA molecules, would act to directly block the translation of group X sPLA2 mRNA by binding thereto and thus preventing protein translation or increasing mRNA degradation, thus decreasing the level of group X sPLA2, and thus activity, in a cell. For example, antisense oligonucleotides of at least about 15 bases and complementary to unique regions of the mRNA transcript sequence encoding group X sPLA2 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention group X sPLA2 gene expression can be reduced by contacting a subject or cell with a small double stranded RNA (dsRNA), or a vector or construct causing the production of a small double stranded RNA, such that group X sPLA2 gene expression is specifically inhibited (i.e. RNA interference or RNAi). Methods for selecting an appropriate dsRNA or dsRNA-encoding vector are well known in the art for genes whose sequence is known (U.S. Pat. Nos. 6,573,099 and 6,506,559; and International Patent Publication Nos. WO 01/36646, WO 99/32619, and WO 01/68836). All or part of the phosphodiester bonds of the siRNAs of the invention are advantageously protected. This protection is generally implemented via the chemical route using methods that are known by art. The phosphodiester bonds can be protected, for example, by a thiol or amine functional group or by a phenyl group. The 5′- and/or 3′-ends of the siRNAs of the invention are also advantageously protected, for example, using the technique described above for protecting the phosphodiester bonds. The siRNAs sequences advantageously comprises at least twelve contiguous dinucleotides or their derivatives.

As used herein, the term “siRNA derivatives” with respect to the present nucleic acid sequences refers to a nucleic acid having a percentage of identity of at least 90% with erythropoietin or fragment thereof, preferably of at least 95%, as an example of at least 98%, and more preferably of at least 98%.

As used herein, “percentage of identity” between two nucleic acid sequences, means the percentage of identical nucleic acid, between the two sequences to be compared, obtained with the best alignment of said sequences, this percentage being purely statistical and the differences between these two sequences being randomly spread over the nucleic acids sequences. As used herein, “best alignment” or “optimal alignment”, means the alignment for which the determined percentage of identity (see below) is the highest. Sequences comparison between two nucleic acids sequences are usually realized by comparing these sequences that have been previously aligned according to the best alignment; this comparison is realized on segments of comparison in order to identify and compare the local regions of similarity. The best sequences alignment to perform comparison can be realized, beside by a manual way, by using the global homology algorithm developed by SMITH and WATERMAN (Ad. App. Math., vol. 2, p: 482, 1981), by using the local homology algorithm developped by NEDDLEMAN and WUNSCH (J. Mol. Biol., vol. 48, p: 443, 1970), by using the method of similarities developed by PEARSON and LIPMAN (Proc. Natl. Acd. Sci. USA, vol. 85, p: 2444, 1988), by using computer softwares using such algorithms (GAP, BESTFIT, BLAST P, BLAST N, FASTA, TFASTA in the Wisconsin Genetics software Package, Genetics Computer Group, 575 Science Dr., Madison, Wis. USA), by using the MUSCLE multiple alignment algorithms (Edgar, Robert C., Nucleic Acids Research, vol. 32, p: 1792, 2004). To get the best local alignment, one can preferably use BLAST software. The identity percentage between two sequences of nucleic acids is determined by comparing these two sequences optimally aligned, the nucleic acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

shRNAs (short hairpin RNA) can also function as inhibitors of gene expression for use in the present invention.

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of group X sPLA2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector as above described.

Alternatively, the animal may be deficient for a gene or a plurality of genes so that said animal cannot produce group X sPLA2s. For example, said animal may be deficient in a gene encoding for group X sPLA2.

Deficient animals, especially deficient mice are generally commercially available. However, methods for producing deficient animal or knock-out animal are well known in the art. For example, Knock-out animals comprising targeted mutations are achieved routinely in the art as provided for example by the method by Joyner, A. L. (Gene Targeting. 1999, Second Edition, The Practical Approach Series, Oxford University Press, New York) and Hogan, B., et al. (Manipulating the mouse embryo. 1994, Second Edition, Cold Spring Harbor Press, Cold Spring Harbor.). For example, the heterozygous and/or homozygous knock-out animal of the above-described methods may be generated by selecting embryonic stem (ES) cell clones carrying a mutated allele for the gene of interest, verifying the targeted mutation in the recombinant embryonic stem cell clones, injecting the verified recombinant embryonic stem cells into blastocysts of wild type animals, transferring these injected blastocysts into pseudo-pregnant foster mothers, breeding chimeras resulting from the blastocysts to wild type animals, testing the offspring resulting from these breedings for the presence of the targeted mutation, breeding heterozygous animals, optionally to generate homozygous knock-out animals. Embryonic stem cells used in the art which may also be used in the methods of this invention comprise for example embryonic stem cells derived from mouse strains such as C57BL/6, BALB/c, DBA/2, CBA/ and SV129. Preferably, embryonic stem cells derived from C57BL/6 mice are used. Methods for producing deficient animals are also described in WO03037074 and US2007056052.

Alternatively, the knock out of the targeted gene may be performed in conditional manner as described in WO2006048466 or WO2006048465, so that the disruption of expression may be performed at a willing time and/or in a specific tissue or cells.

The animal according to the invention may be selected from the group consisting of rat, mouse, cow, pig, horse, chicken, cat, and dog. More preferably, the animal is a mouse. Said mouse may be selected from the groups consisting of C57BL/6, BALB/c, DBA/2, CBA/and SV129 strains.

In one embodiment, the animal may be deficient in another gene to favour atherosclerosis development in said animal. For example, the animal may be further deficient for LDL receptor or apolipoprotein E.

A further aspect of the invention relates to use of a non human deficient animal for group X sPLA2 as a model for atherosclerosis.

Such animal model may be useful for screening drugs useful for treating atherosclerosis.

A further aspect of the invention relates to a method for screening a drug for treating a atherosclerosis, said method comprising the step consisting of:

i) providing a non human animal model for atherosclerosis according to the invention

ii) administering to said animal a candidate compound

iii) measuring the effect of said compound on atherosclerosis in said animal.

iv) selecting the candidate compound capable of reducing said atherosclerosis in said animal.

Methods for measuring atherosclerosis in an animal are well known in the art and may be performed as described in the EXAMPLE 1.

According to a one embodiment of the invention, the candidate compound may be selected from a library of compounds previously synthesised, or a library of compounds for which the structure is determined in a database, or from a library of compounds that have been synthesised de novo. The candidate compound may be selected from the group of (a) proteins or peptides, (b) nucleic acids and (c) organic or chemical compounds. Illustratively, libraries of pre-selected candidate nucleic acids may be obtained by performing the SELEX method as described in documents U.S. Pat. No. 5,475,096 and U.S. Pat. No. 5,270,163.

Diagnostics Methods According to the Invention:

A further aspect of the invention relates to a method of testing a subject thought to have or be predisposed to having atherosclerosis, which comprises the step of analyzing a biological sample from said subject for:

(i) detecting the presence of a mutation in the gene encoding for group X sPLA2 and/or its associated promoter, and/or

(ii) analyzing the expression of the gene encoding for group X sPLA2 and/or

(iii) analysing the activity of group X sPLA2

As used herein, the term “biological sample” refers to any sample from a subject such as blood or serum.

Typical techniques for detecting a mutation in the gene encoding for group X sPLA2 may include restriction fragment length polymorphism, hybridisation techniques, DNA sequencing, exonuclease resistance, microsequencing, solid phase extension using ddNTPs, extension in solution using ddNTPs, oligonucleotide assays, methods for detecting single nucleotide polymorphism such as dynamic allele-specific hybridisation, ligation chain reaction, mini-sequencing, DNA “chips”, allele-specific oligonucleotide hybridisation with single or dual-labelled probes merged with PCR or with molecular beacons, and others.

Analyzing the expression of the gene encoding for group X sPLA2 may be assessed by any of a wide variety of well-known methods for detecting expression of a transcribed nucleic acid or translated protein.

In a preferred embodiment, the expression of the gene encoding for group X sPLA2 is assessed by analyzing the expression of mRNA transcript or mRNA precursors, such as nascent RNA, of said gene. Said analysis can be assessed by preparing mRNA/cDNA from cells in a biological sample from a subject, and hybridizing the mRNA/cDNA with a reference polynucleotide. The prepared mRNA/cDNA can be used in hybridization or amplification assays that include, but are not limited to, Southern or Northern analyses, polymerase chain reaction analyses, such as quantitative PCR (TaqMan), and probes arrays such as GeneChip™ DNA Arrays (AFF YMETRIX).

Advantageously, the analysis of the expression level of mRNA transcribed from the gene encoding for group X sPLA2 involves the process of nucleic acid amplification, e. g., by RT-PCR (the experimental embodiment set forth in U.S. Pat. No. 4,683, 202), ligase chain reaction (BARANY, Proc. Natl. Acad. Sci. USA, vol. 88, p: 189-193, 1991), self sustained sequence replication (GUATELLI et al., Proc. Natl. Acad. Sci. USA, vol. 57, p: 1874-1878, 1990), transcriptional amplification system (KWOH et al., 1989, Proc. Natl. Acad. Sci. USA, vol. 86, p: 1173-1177, 1989), Q-Beta Replicase (LIZARDI et al., Biol. Technology, vol. 6, p: 1197, 1988), rolling circle replication (U.S. Pat. No. 5,854, 033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In another preferred embodiment, the expression of the gene encoding for group X sPLA2 is assessed by analyzing the expression of the protein translated from said gene. Said analysis can be assessed using an antibody (e.g., a radio-labeled, chromophore-labeled, fluorophore-labeled, or enzyme-labeled antibody), an antibody derivative (e.g., an antibody conjugate with a substrate or with the protein or ligand of a protein of a protein/ligand pair (e.g., biotin-streptavidin)), or an antibody fragment (e.g., a single-chain antibody, an isolated antibody hypervariable domain, etc.) which binds specifically to the protein translated from the gene encoding for group X sPLA2.

Said analysis can be assessed by a variety of techniques well known from one of skill in the art including, but not limited to, enzyme immunoassay (EIA), radioimmunoassay (RIA), Western blot analysis and enzyme linked immunoabsorbant assay (RIA).

The method of the invention may comprise comparing the level of expression of the gene encoding for group X sPLA2 in a biological sample from a subject with the normal expression level of said gene in a control. A significantly weaker level of expression of said gene in the biological sample of a subject as compared to the normal expression level is an indication that the patient has or is predisposed to developing atherosclerosis. The “normal” level of expression of the gene encoding for group X sPLA2 is the level of expression of said gene in a biological sample of a subject not afflicted by any disease associated with an atherosclerosis. Preferably, said normal level of expression is assessed in a control sample (e.g., sample from a healthy subject, which is not afflicted by any disease associated with an atherosclerosis and preferably, the average expression level of said gene in several control samples.

According to the invention, the measure of group X sPLA2 activity can be performed by a fluorimetric assay according to Radvanyi et al. (1989 Anal Biochem 177: 103-9) as modified by Pernas et al. (1991 Biochem Biophys Res Commun 178: 1298-1305). Typically group X sPLA2 activity may be also determined by a process based on a fluorimetric assay comprising contacting the biological sample containing said group X sPLA2 and taken from said patient, with a substrate at a various concentration and measuring the flurorescence intensity that results from the hydrolysis of the substrate.

The invention will be further illustrated by the following examples. However, these examples should not be interpreted in any way as limiting the scope of the present invention.

### EXAMPLE 1

### Methods

The hearts of mice were removed and successive 10-μm transversal sections of aortic sinus were obtained. Lipids and collagen were detected using Oil red O and Sirius red stainings, respectively. The presence of macrophages, T lymphocytes, and smooth muscle cells were determined using specific antibodies, as previously described {Mallat, 2003}. Stringent TUNEL (ApopDETEK kit, ENZO Diagnostics) was performed to detect apoptotic cells. At least 4 sections per mouse were examined for each immunostaining, and appropriate negative controls will be performed. Morphometric studies were made using the Histolab software from Microvisions {Mallat, 2003}. Isolation and stimulation of splenocytes or T cells were performed as previously described [Ait-Oufella, 2007].

### EXAMPLE 2

### Group X PLA2 Deficiency in Bone Marrow-Derived Cells Accelerates Atherosclerosis

We performed bone marrow transplantation experiments with mouse group X knock-out mice in LDL receptor knock-out mice and observed that deficiency of mouse group X sPLA2 in leukocytes unexpectedly results in increased atherosclerosis. For those experiments, bone marrow cells harvested from C57BL/6 group X sPLA2(−/−) and group X sPLA2(+/+) mice were transplanted into lethally irradiated (9.5 Gy) male LDL receptor(−/−) mice. Six weeks after transplantation, mice were fed a high-fat cholate-free diet for 8 weeks. Despite no differences in weights and serum cholesterol levels between the 2 groups, mouse group X deficiency in leukocytes induced a >2-fold increase in lesion development in the aortic sinus compared with controls.

### EXAMPLE 3

### Group X sPLA2 Deficiency in Bone Marrow-Derived Cells Enhances Mononuclear Cell Adhesion to Atherosclerotic Arteries and Promotes Lesional T Cell Accumulation

We next examined lesion composition. Interestingly, we found a marked increase in T cell accumulation in lesions of Ldlr/group X sPLA2−/− mice despite no difference in blood or spleen T cell count.

Therefore, we examined the effect of group X sPLA2 deficiency on the adherence of peripheral blood mononuclear cells to activated carotid vessels. Interestingly, we found that blood mononuclear cells from mouse group X deficient mice adhere more tightly to endothelial cells or isolated fresh carotids retrieved from LDL receptor knock-out mice, potentially explaining increased recruitment to and accumulation within the lesions of group X sPLA2−/− mice.

### EXAMPLE 4

### Group X sPLA2 Deficiency in Bone Marrow-Derived Cells Promotes a Pro-Atherogenic TH1 Cell Response

We next addressed the cytokine profile of the T cell response in group X sPLA2−/− mice. Interestingly, we found that activated splenocytes from mouse group X deficient mice in both WT or LDL receptor knock-out genotypes showed increased IL12 production compared with WT control splenocytes and this was associated with a marked Th2 phenotype, as revealed by enhanced production of IFN-γ by CD4+ T lymphocytes.

### EXAMPLE 5

### Group X sPLA2 Deficiency Enhances Apoptotic Cell Accumulation within the Lesions and Promotes the Development of Large Necrotic Cores

group X sPLA2 generates high levels of lysophosphatidylcholine, a potent chemo-attractant released by apoptotic cells to promote their removal by macrophages. Thus, we hypothesized that group X sPLA2 deficiency may lead to apoptotic cell accumulation within the lesions and may therefore favour the formation of large secondary necrotic lipid cores. Indeed, we found a marked increase in TUNEL-positive apoptotic cell material within the lesions of Ldlr−/−/group X sPLA2−/− mice associated with a 3-fold increase in the size of the necrotic core.

### REFERENCES

Throughout this application, various references describe the state of the art to which this invention pertains. The disclosures of these references are hereby incorporated by reference into the present disclosure.

Ait-Oufella H, Kinugawa K, Zoll J, Simon T, Boddaert J, Heeneman S, Blanc-Brude O, Barateau V, Potteaux S, Merval R, Esposito B, Daemen M, Lesèche G, Boulanger C M, Tedgui A, Mallat Z. Lactadherin-dependent phagocytosis of apoptotic cells maintains a protective regulatory immune response in atherosclerosis. Circulation, 2007, 115:2168-2177.

Bostrom, M. A., Boyanovsky, B. B., Jordan, C. T., Wadsworth, M. P., Taatjes, D. J., de Beer, F. C., and Webb, N. R. (2007) Group v secretory phospholipase A2 promotes atherosclerosis: evidence from genetically altered mice, Arterioscler. Thromb. Vasc. Biol. 27, 600-606.

Boyanovsky, B. B., and Webb, N. R. (2008) Biology of Secretory Phospholipase A2, Cardiovasc. Drugs Ther. in press.

Boyanovsky, B., Zack, M., Forrest, K., and Webb, N. R. (2009) The Capacity of Group V sPLA2 to Increase Atherogenicity of ApoE−/− and LDLR−/− Mouse LDL In Vitro Predicts its Atherogenic Role In Vivo, Arterioscler. Thromb. Vasc. Biol. in press.

Cummings, B. S. (2007) Phospholipase A2 as targets for anti-cancer drugs, Biochem. Pharmacol. 74, 949-959.

Curfs, D. M., Ghesquiere, S. A., Vergouwe, M. N., van der Made, I., Gijbels, M. J., Greaves, D. R., Verbeek, J. S., Hofker, M. H., and de Winther, M. P. (2008)

Fujioka, D., Saito, Y., Kobayashi, T., Yano, T., Tezuka, H., Ishimoto, Y., Suzuki, N., Yokota, Y., Nakamura, T., Obata, J. E., Kanazawa, M., Kawabata, K., Hanasaki, K., and Kugiyama, K. (2008) Reduction in myocardial ischemia/reperfusion injury in group X secretory phospholipase A2-deficient mice, Circulation 117, 2977-2985.

Ghesquiere, S. A., Gijbels, M. J., Anthonsen, M., van Gorp, P. J., van der Made, I., Johansen, B., Hofker, M. H., and de Winther, M. P. (2005) Macrophage-specific overexpression of group IIa sPLA2 increases atherosclerosis and enhances collagen deposition, J. Lipid Res. 46, 201-210.

Ghesquiere, S. A., Hofker, M. H., and de Winther, M. P. (2005) The role of phospholipases in lipid modification and atherosclerosis, Cardiovasc. Toxicol. 5, 161- 182.

Ivandic, B., Castellani, L. W., Wang, X. P., Qiao, J. H., Mehrabian, M., Navab, M., Fogelman, A. M., Grass, D. S., Swanson, M. E., de Beer, M. C., de Beer, F., and Lusis, A. J. (1999) Role of group II secretory phospholipase A2 in atherosclerosis: 1. Increased atherogenesis and altered lipoproteins in transgenic mice expressing group IIa phospholipase A2, Arterioscler. Thromb. Vasc. Biol. 19, 1284-1290.

Jonsson-Rylander, A. C., Lundin, S., Rosengren, B., Pettersson, C., and Hurt-Camejo, E. (2008) Role of secretory phospholipases in atherogenesis, Curr. Atheroscler. Rep. 10, 252-259.

Karabina, S. A., Brocheriou, I., Le Naour, G., Agrapart, M., Durand, H., Gelb, M., Lambeau, G., and Ninio, E. (2006) Atherogenic properties of LDL particles modified by human group X secreted phospholipase A2 on human endothelial cell function, Faseb J. 20, 2547-2549.

Koenig, W., and Khuseyinova, N. (2008) Lipoprotein-Associated and Secretory Phospholipase A(2) in Cardiovascular Disease: The Epidemiological Evidence, Cardiovascular drugs and therapy/sponsored by the International Society of Cardiovascular Pharmacotherapy in press.

Mallat, Z., Benessiano, J., Simon, T., Ederhy, S., Sebella-Arguelles, C., Cohen, A., Huart, V., Wareham, N. J., Luben, R., Khaw, K. T., Tedgui, A., and Boekholdt, S. M. (2007) Circulating secretory phospholipase A2 activity and risk of incident coronary events in healthy men and women: the EPIC-Norfolk study, Arterioscler. Thromb. Vasc. Biol. 27, 1177 1183.

Mallat, Z., Steg, P. G., Benessiano, J., Tanguy, M. L., Fox, K. A., Collet, J. P., Dabbous, O. H., Henry, P., Carruthers, K. F., Dauphin, A., Arguelles, C. S., Masliah, J., Hugel, B., Montalescot, G., Freyssinet, J. M., Asselain, B., and Tedgui, A. (2005) Circulating secretory phospholipase A2 activity predicts recurrent events in patients with severe acute coronary syndromes, J. Am. Coll. Cardiol. 46, 1249-1257.

Menschikowski, M., Hagelgans, A., and Siegert, G. (2006) Secretory phospholipase A2 of group IIA: is it an offensive or a defensive player during atherosclerosis and other inflammatory diseases?, Prostaglandins Other Lipid Mediat. 79, 1-33.

Nevalainen, T. J., Haapamaki, M. M., and Gronroos, J. M. (2000) Roles of secretory phospholipases A(2) in inflammatory diseases and trauma, Biochim. Biophys. Acta 1488, 83-90.

Reid, R. C. (2005) Inhibitors of secretory phospholipase A2 group IIA, Curr. Med. Chem. 12, 3011-3026.

Rosenson, R. S. (2009) Future role for selective phospholipase A2 inhibitors in the prevention of atherosclerotic cardiovascular disease, Cardiovasc. Drugs Ther. 23, 93-101.

Sato, H., Kato, R., Isogai, Y., Saka, G., Ohtsuki, M., Taketomi, Y., Yamamoto, K., Tsutsumi, K., Yamada, J., Masuda, S., Ishikawa, Y., Ishii, T., Kobayashi, T., Ikeda, K., Taguchi, R., Hatakeyama, S., Hara, S., Kudo, I., Itabe, H., and Murakami, M. (2008) Analyses of group III secreted phospholipase A2 transgenic mice reveal potential participation of this enzyme in plasma lipoprotein modification, macrophage foam cell formation, and atherosclerosis, J. Biol. Chem. 283, 33483-33497.

Scott, K. F., Graham, G. G., and Bryant, K. J. (2003) Secreted phospholipase A2 enzymes as therapeutic targets, Expert Opin. Ther. Targets 7, 427-440.

Tietge, U. J., Pratico, D., Ding, T., Funk, C. D., Hildebrand, R. B., Van Berkel, T., and Van Eck, M. (2005) Macrophage-specific expression of group IIA sPLA2 results in accelerated atherogenesis by increasing oxidative stress, J. Lipid Res. 46, 1604-1614.

Vadas, P., Browning, J., Edelson, J., and Pruzanski, W. (1993) Extracellular Phospholipase A2 expression and inflammation: the relationship with associated disease states, J. Lip. Mediat. 8, 1-30.

Webb, N. R., Bostrom, M. A., Szilvassy, S. J., van der Westhuyzen, D. R., Daugherty, A., and de Beer, F. C. (2003) Macrophage-expressed group IIA secretory phospholipase A2 increases atherosclerotic lesion formation in LDL receptor-deficient mice, Arterioscler. Thromb. Vasc. Biol. 23, 263-268.

1-4. (canceled)

5. A non human animal model for atherosclerosis wherein said atherosclerosis is induced by inhibiting the expression or activity of group X sPLA2.

6. A method of testing a subject thought to have or be predisposed to having atherosclerosis, which comprises the step of
analyzing a biological sample from said subject by:
(i) detecting the presence of a mutation in a gene encoding for a group X sPLA2 and/or its associated promoter, and/or
(ii) analyzing the expression of the gene encoding for group X sPLA2 and/or
(iii) analysing the activity of the group X sPLA2.

7. A method of treating atherosclerosis in a patient in need thereof comprising
administering to said patient a therapeutically effective amount of a group X sPLA2 polypeptide.

8. A method of treating atherosclerosis in a patient in need thereof comprising
administering to said patient a therapeutically effective amount of a nucleic acid molecule encoding a group X sPLA2 polypeptide.

9. A method of treating atherosclerosis in a patient in need thereof comprising
administering to said patient a therapeutically effective amount of a vector comprising a nucleic acid molecule encoding a group X sPLA2 polypeptide.

10. A method of treating atherosclerosis in a patient in need thereof comprising
administering to said patient a therapeutically effective amount of a host cell comprising
a nucleic acid molecule encoding a group X sPLA2 polypeptide or
a vector comprising a nucleic acid encoding a group X sPLA2 polypeptide.
